(19) United States
(12) Patent Application Publication
(10) Pub. No.: US 2021/0212667 A1
(43) Pub. Date: Jul. 15, 2021

(54) HIGH-INTENSITY FOCUSED ULTRASOUND THERAPEUTIC SYSTEM AND REAL-TIME MONITORING METHOD THEREOF

(71) Applicant: National Taiwan University of Science and Technology, Taipei (TW)

(72) Inventor: Che-Chou Shen, Taipei (TW)

(73) Assignee: National Taiwan University of Science and Technology, Taipei (TW)

(21) Appl. No.: 17/013,873

(22) Filed: Sep. 8, 2020

(30) Foreign Application Priority Data
Jan. 14, 2020 (TW) 109101138
Jul. 30, 2020 (TW) 109125701

(51) Int. Cl. A61B 8/08; A61B 8/14

(52) U.S. Cl. CPC A61B 8/5269; A61B 2017/00106; A61B 8/14

(57) ABSTRACT

A high-intensity focused ultrasound (HIFU) therapeutic system includes a first ultrasonic transmitter and an ultrasonic imaging apparatus. The first ultrasonic transmitter transmits a HIFU therapeutic signal to a target. The ultrasonic imaging apparatus includes a second ultrasonic transmitter, an echo receiver, and a signal processor. The second ultrasonic transmitter alternately transmits a first imaging signal and a second imaging signal to the target, and the two form a complementary Golay code pair, where a bit period of a Golay code is determined by a transmission frequency of HIFU. The echo receiver receives a first echo signal, a second echo signal, and an interference signal. The signal processor performs a decoding operation on the first echo signal and the second echo signal and suppresses the interference signal to generate a high-quality ultrasonic image for monitoring a HIFU therapy.

### CROSS-REFERENCE TO RELATED APPLICATION

This application claims the priority benefits of Taiwan application serial no. 109101138, filed on Jan. 14, 2020, and Taiwan application serial no. 109125701, filed on Jul. 30, 2020. The entirety of each of the above-mentioned patent applications is hereby incorporated by reference herein and made a part of this specification.

### BACKGROUND

### Technical Field

The disclosure relates to an ultrasonic therapeutic system and a real-time monitoring method thereof, and in particular, to a high-intensity focused ultrasound (HIFU) therapeutic system and a real-time monitoring method thereof.

### Description of Related Art

HIFU is a non-invasive therapeutic technology featuring a strong focusing characteristic. High acoustic energy is introduced from the outside of the body to destroy tissues and to ablate a small volume of a specific part of the body, resulting in coagulation and necrosis of a target tissue volume without harming surrounding healthy tissues and organs. Therefore, a HIFU knife is widely used in current non-invasive therapeutic fields, such as a tumour and cancer therapy, hemostasis, blood-brain barrier opening, and other non-invasive therapies.

During a HIFU ablation therapy, a monitoring image is required to assist the therapy. Both magnetic resonance imaging (MRI) and ultrasound imaging may be used for HIFU therapy guidance. An advantage of MR-gHIFU is that a temperature change of a tissue may be measured and arbitrary cross-sectional image information in a three-dimensional space may be provided. However, an MR apparatus is bulky, expensive, and requires a long image collection time, and therefore, it is impossible to monitor a HIFU therapy process in real time.

On the contrary, US-gHIFU has potential for real-time monitoring during a therapy together with higher time resolution, higher portability, and lower costs. However, HIFU backscattering will cause strong interference patterns on the entire ultrasound image, making it difficult to monitor a change in tissue ablation in real time. This becomes one of challenges of US-gHIFU implementation.

### SUMMARY

The disclosure provides a high-intensity focused ultrasound (HIFU) therapeutic system and a real-time monitoring method thereof to improve an image signal-to-noise ratio and suppress the HIFU interference signal during ultrasound imaging.

A HIFU therapeutic system of the disclosure includes a first ultrasonic transmitter and an ultrasonic imaging apparatus. The first ultrasonic transmitter is configured to transmit a HIFU therapeutic signal to a target. The ultrasonic imaging apparatus includes a second ultrasonic transmitter, an echo receiver, and a signal processor. The second ultrasonic transmitter is configured to alternately transmit a first imaging signal and a second imaging signal to the target, where the first imaging signal and the second imaging signal form a complementary Golay code pair. The echo receiver is configured to receive a first echo signal corresponding to the first imaging signal, a second echo signal corresponding to the second imaging signal, and an interference signal caused by the HIFU therapeutic signal. The signal processor is configured to perform a decoding operation on the first echo signal, the second echo signal, and the interference signal to generate an imaging signal and a suppressed interference signal.

The real-time monitoring method of the HIFU therapeutic system of the disclosure includes the following steps. A HIFU therapeutic signal is transmitted to a target. A first imaging signal and a second imaging signal are alternatively transmitted to the target, where the first imaging signal and the second imaging signal form a complementary Golay code pair. A first echo signal corresponding to the first imaging signal, a second echo signal corresponding to the second imaging signal, and an interference signal caused by the HIFU therapeutic signal are received. A decoding operation is performed on the first echo signal, the second echo signal, and the interference signal to generate an imaging signal and a suppressed interference signal.

Based on the above, according to the disclosure, ultrasonic imaging may be performed by transmitting a Golay code pair to improve the image signal-to-noise ratio. Further, according to the disclosure, an interference signal may be suppressed to eliminate the interference pattern caused by a HIFU signal on the monitoring ultrasonic image. Therefore, through the technology provided by the disclosure, real-time ultrasound image guidance with high image quality is provided at no cost of the monitoring window size while the efficiency of HIFU therapy is maintained as well.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A is a schematic diagram illustrating signal transmission of a high-intensity focused ultrasound (HIFU) therapeutic system according to the disclosure.

FIG. 1B is a schematic diagram illustrating another exemplary configuration of ultrasonic probes according to an embodiment of the disclosure.

FIG. 1C is a schematic diagram illustrating signal receiving of the HIFU therapeutic system according to the disclosure.

FIG. 2 is a schematic diagram illustrating decoding of echo signals Â and \hat{B}.

FIG. 3A is a diagram illustrating a correspondence between a HIFU interference signal R_HIFU and the echo signals Â and \hat{B} according to the first embodiment of the disclosure.

FIG. 3B is a diagram illustrating a correspondence between the HIFU interference signal R_HIFU and the echo signals Â and \hat{B} according to the second embodiment of the disclosure.

FIG. 4A is a schematic diagram illustrating decoding details of the HIFU interference signal in the first embodiment.

FIG. 4B is a schematic diagram illustrating decoding details of the HIFU interference signal in the second embodiment.

FIG. 5 is a flow chart illustrating steps of a real-time monitoring method of a HIFU therapeutic system.

FIG. 6A is a schematic diagram illustrating comparison before and after Golay decoding for the first embodiment.

FIG. 6B is a schematic diagram illustrating comparison before and after Golay decoding for the second embodiment.

### DESCRIPTION OF THE EMBODIMENTS

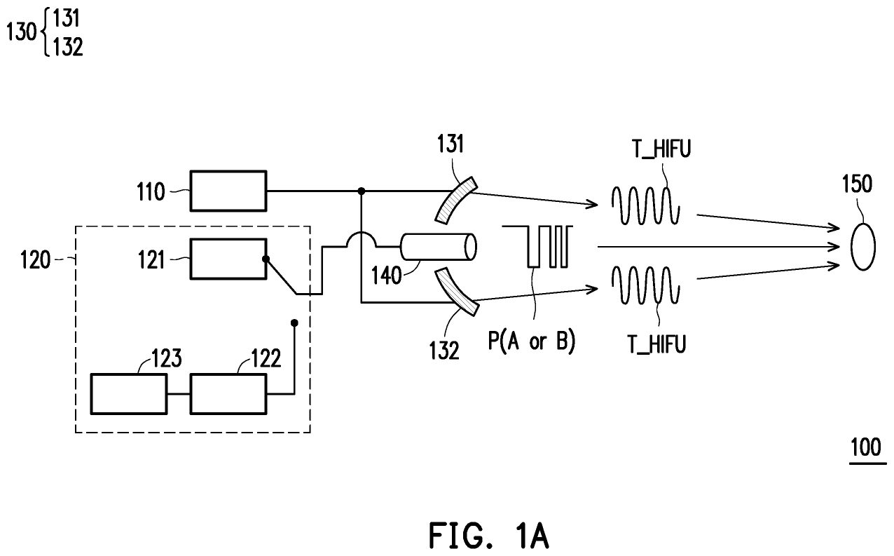

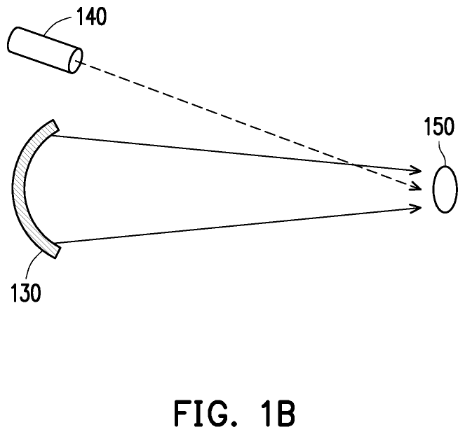

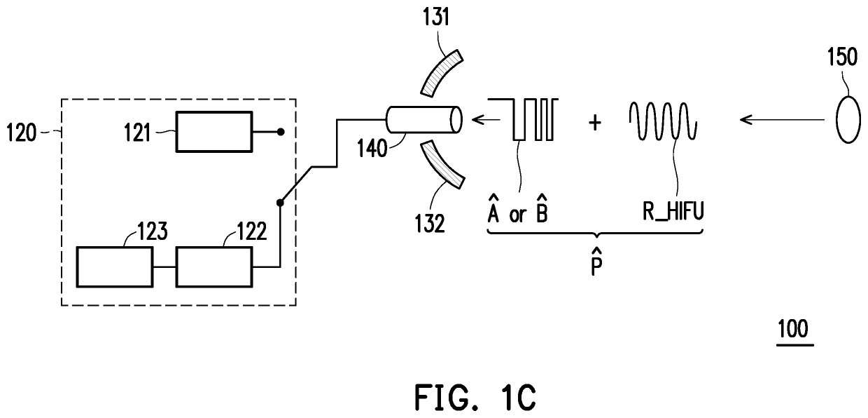

FIG. 1A is a schematic diagram illustrating signal transmission of a high-intensity focused ultrasound (HIFU) system according to the disclosure. Referring to FIG. 1A, a HIFU therapeutic system 100 includes a HIFU signal transmitter 110, an imaging apparatus 120, a probe 130, and a probe 140. The HIFU signal transmitter 110 is coupled to the probe 130 to generate a HIFU signal T_HIFU. The HIFU signal T_HIFU is transmitted to a target 150 through the probe 130 to perform a therapy on the target 150. The probe 130 may include therapeutic arrays 131 and 132. The imaging apparatus 120 includes an imaging signal transmitter 121, an echo receiver 122, and a signal processor 123. The imaging signal transmitter 121 may generate an encoded waveform as an imaging signal P to be transmitted to the target 150 through the probe 140. The target 150 is the volume for HIFU ablation therapy.

In FIG. 1A, an orientation of the probe 130 is the same as an orientation of the probe 140. However, the orientation of the probe 130 may also be different from the orientation of the probe 140. FIG. 1B is a schematic diagram illustrating another exemplary configuration of ultrasonic probes according to an embodiment of the disclosure. It should be noted that, in order to conveniently indicate relative positions of the probe 130, the probe 140, and the target 150, FIG. 1B only briefly depicts the probe 130, the probe 140, and the target 150, and other components are omitted. Referring to FIG. 1B, the probe 140 is positioned on one side of the probe 130. The orientation of the probe 130 is different from the orientation of the probe 140. In fact, the probe 140 can be arbitrarily positioned in the disclosure, as long as its orientation is aligned with the target 150 for monitoring.

FIG. 1C is a schematic diagram illustrating signal receiving of the HIFU therapeutic system according to the disclosure. It should be noted that, in order to conveniently show a signal transmission path, FIG. 1C only briefly depicts the imaging apparatus 120, the probe 130, the probe 140, and the target 150, and the rest of the components are omitted. Referring to FIG. 1C, the echo receiver 122 receives an echo signal \hat{P} through the probe 140, and the echo signal \hat{P} includes a reflected encoded signal and a HIFU interference signal R_HIFU caused by backscattering of the HIFU signal T_HIFU. The echo signal \hat{P} is then decoded by the signal processor 123. For each element in FIG. 1C, refer to the descriptions of the element with the same name in FIG. 1A. The descriptions thereof are omitted herein.

It should be particularly noted that the imaging signal P sent by the imaging signal transmitter 121 is a complementary Golay code pair, including imaging signals A and B. The imaging signal P is mainly a 4^{N}-bit Golay code, and N is a positive integer. The purpose is to maintain image resolution while providing a better signal-to-interference ratio (SIR). The imaging signal P in the present embodiment is mainly a 4-bit Golay code. The imaging signal transmitter 121 is excited by a Golay code to alternately send the signals A and B. The echo receiver 122 receives the corresponding echo signal Â, the corresponding echo signal \hat{B}, and the HIFU interference signal R_HIFU. The echo signal is then decoded by the signal processor 123. It should be noted that, when the imaging signal is a 4-bit Golay code, the echo signal is also a 4-bit Golay code. Specifically, the waveform of the echo signal Â and \hat{B} is the same as the waveform of the imaging signal A and B, respectively.

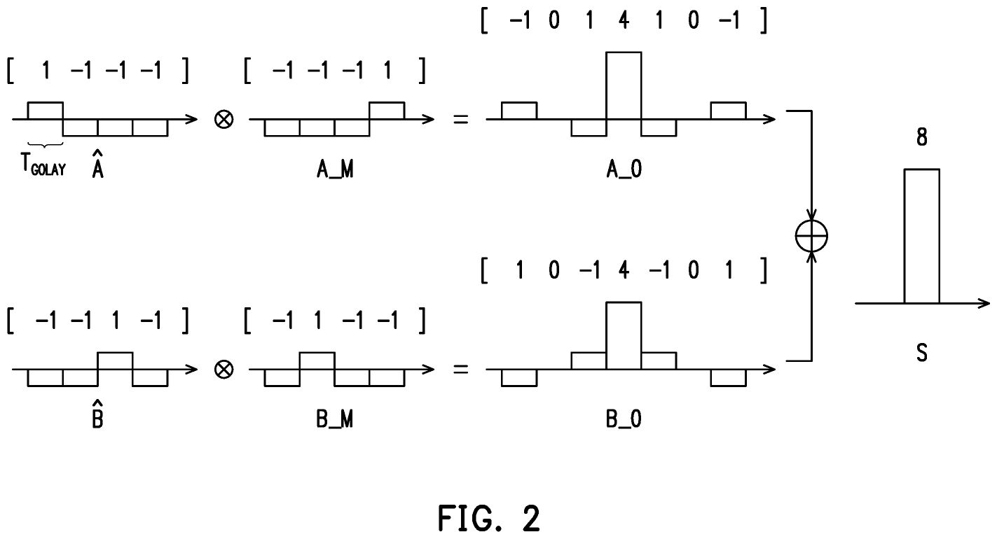

FIG. 2 is a schematic diagram illustrating decoding of echo signals Â and \hat{B}. Referring to FIG. 2, A_O represents an output signal generated by a convolution operation performed on the echo signal Â through a matching filter A_M, and B_O represents an output signal generated by the convolution operation performed on the echo signal \hat{B} through a matching filter B_M. S represents a complementary sum signal generated by an addition of the output signals A_O and B_O. The signal processor 123 respectively performs the convolution operation on the echo signals Â and \hat{B} through the corresponding matching filters A_M and B_M, and adds calculation results to generate the complementary sum signal S. A calculation formula of the signal processor 123 may be expressed as:

Ã₁(n)⊗A₁(−n)+\tilde{B}₁(n)⊗B₁(−n)=2Nδ(n) (1)

The echo signals Â and \hat{B} are respectively represented as Ã₁(n) and \tilde{B}₁(n). The matching filters A_M and B_M are respectively expressed as A₁(−n) and B₁(−n). In fact, the matching filters A_M and B_M are respectively the time reversal of the encoded waveform of the imaging signal A and B. ⊗ represents the convolution operation, and + represents the addition operation. N is the number of bits, and δ(n) represents a main lobe signal in the range direction. It may be learned from formula (1) that energy of the main lobe signal is increased by 2N times after being processed by the signal processor 123.

In the present embodiment, the 4-bit echo signal Ã is, for example, [1 −1 −1 −1], and the corresponding matching filter A_M is, for example, [−1 −1 −1 1] (a time reversal of [1 −1 −1 −1]). The echo signal \hat{B} is, for example, [−1 −1 1 −1], and the corresponding matching filter B_M is, for example, [−1 1 −1 −1] (a time reversal of [−1 −1 1 −1]). The numbers in “[]”, represent a sequence of bit waveform. In detail, taking the echo signal Ã as an example, the echo signal Ã includes four bit waveforms in total, whose phases of the bit waveforms are one positive phase and three reversed phases in sequence. A time length T_{Golay} of the bit waveform is defined as a bit period. For any specific T_{Golay}, a designer may select an operating frequency and a cycle number of the imaging signal to meet the imaging requirement.

Referring to FIG. 2 again, the convolution operation may be performed on the echo signal Ã ([1 −1 −1 −1]) through the corresponding matching filter A_M ([−1 −1 −1 1]) to obtain an output signal A_O ([−1 0 1 4 1 0 −1]). The convolution operation may be performed on the echo signal \tilde{B} ([−1 −1 1 −1]) through the corresponding matching filter B_M ([−1 1 −1 −1]) to obtain an output signal B_O ([1 0 −1 4 −1 0 1]). It may be learned that an amplitude of the main lobe signal in the output signals A_O and B_O is increased to 4, and a side lobe signal ([−1 0 1 1 0 −1]) in the output signal A_O and a side lobe signal ([1 0 −1 −1 0 1]) in B_O have opposite bit waveforms. Therefore, after the output signal A_O and the output signal B_O are added, the amplitude of the main lobe signal is increased to 8, while the side lobe signal is cancelled. Briefly, the echo signals Â and \hat{B} are processed by the signal processor 123 to enhance the main lobe signal and completely eliminate the side lobe signal at the same time, achieving a pulse compression effect.

In the first embodiment of the disclosure, a ratio of a Golay code bit period of an imaging signal to a HIFU signal period is specified, as shown in formula (2):

$${T}_{G o l a y} = \frac{2 a + 1}{4} {T}_{HIFU} \qquad ( 2 )$$

Herein, T_{Golay} represents the bit period of the imaging signal (Golay code), T_{HIFU} represents the HIFU signal period, and a represents a natural number. When a is 0, T_{Golay}=¼*T_{HIFU}, indicating that the Golay code bit period of the imaging signal is ¼ (0.25) times the HIFU signal period. When a is 1, T_{Golay}=¾*T_{HIFU}, indicating that the Golay code bit period of the imaging signal is 3/4 (0.75) times the HIFU signal period. When a is 2, T_{Golay}= 5/4*T_{HIFU}, indicating that the Golay code bit period of the imaging signal is 5/4 (1.25) times the HIFU signal period. A case in which a is greater than or equal to 3 may be deduced by analogy.

In the second embodiment of the disclosure, a ratio of a Golay code bit period of an imaging signal to a HIFU signal period is specified, as shown in formula (3):

$${T}_{G o l a y} = \frac{2 a + 2}{4} {T}_{H I F U} \qquad ( 3 )$$

When a is 0, T_{Golay}= 2/4*T_{HIFU}, indicating that the Golay code bit period of the imaging signal is 2/4 (0.5) times the HIFU signal period. When a is 1, T_{Golay}= 4/4*T_{HIFU}, indicating that the Golay code bit period of the imaging signal is 4/4 (1.0) times the HIFU signal period. When a is 2, T_{Golay}= 6/4*T_{HIFU}, indicating that the Golay code bit period of the imaging signal is 6/4 (1.5) times the HIFU signal period. A case in which a is greater than or equal to 3 may be deduced by analogy.

In other words, the selection of a value determines the Golay code bit period of the imaging signal, and therefore determines an operating frequency of the imaging signal. When an operating frequency of an ultrasound imaging signal is lowered, a greater image penetration depth may be obtained. When an operating frequency of an ultrasound imaging signal is raised, a higher image resolution may be obtained.

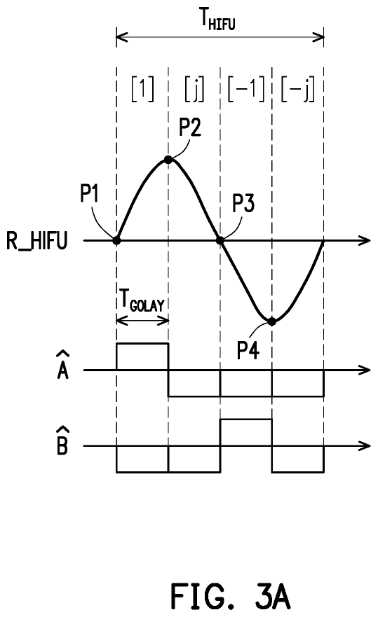

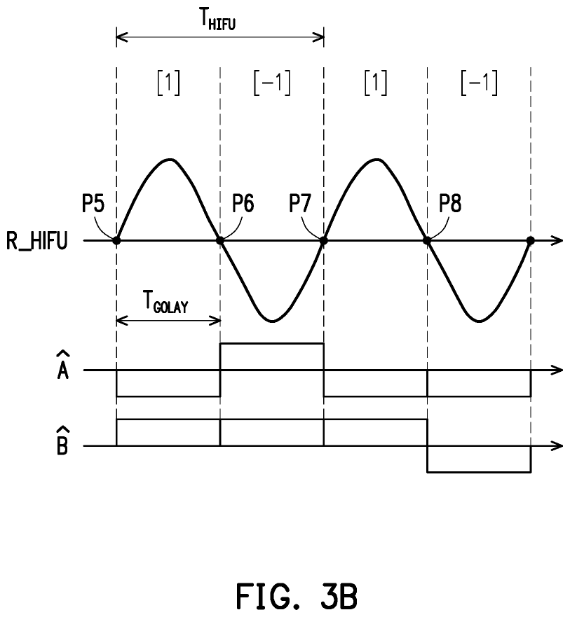

FIG. 3A is a diagram illustrating a correspondence between a HIFU interference signal R_HIFU and the echo signals Â and \hat{B} according to a first embodiment of the disclosure. Referring to FIG. 3A, T_{Golay} of the echo signals Â and \hat{B} is ¼ (0.25) times T_{HIFU} of the HIFU interference signal. In this case, the bit sequence of the echo signals Â and \hat{B} corresponds to phase angles 0°, 90°, 180°, and 270° of the HIFU interference signal (respectively indicated as P1 to P4). Therefore, for the four bits of the echo signals Â and \hat{B}, the corresponding HIFU interference signal may be represented as sequences [1], [j], [−1] and [−j] in sequence. As shown in FIG. 3A, codes of the echo signals Â and \hat{B} in the present embodiment are respectively [1 −1 −1 −1] and [−1 −1 1 −1].

FIG. 3B is a diagram illustrating a correspondence between the HIFU interference signal R_HIFU and the echo signals A and A according to a second embodiment of the disclosure. Referring to FIG. 3B, T_{Golay} of the echo signals Â and \hat{B} is ½ (0.5) times T_{HIFU} of the HIFU interference signal. In this case, the bit sequence of the echo signals Â and \hat{B} corresponds to phase angles 0°, 180°, 360°, and 540° of the HIFU interference signal (respectively indicated as P5 to P8). Therefore, for the four bits of the echo signals Â and \hat{B} , the corresponding HIFU interference signal may be represented as sequences [1], [−1], [1] and [−1] in sequence. As shown in FIG. 3B, codes of the echo signals Â and \hat{B} in the present embodiment are respectively [−1 1 −1 −1] and [1 1 1 −1].

Referring to FIG. 1 again, the imaging signal transmitter 121 of the imaging apparatus 120 may trigger the HIFU signal transmitter 110 to transmit the HIFU signal T_HIFU and vice versa, thereby achieving synchronization between the echo signals Â and \hat{B} and the HIFU interference signal R_HIFU. Due to synchronization between imaging and a therapy, sequences of HIFU interference signals received are the same for both the echo signals Â and \hat{B}.

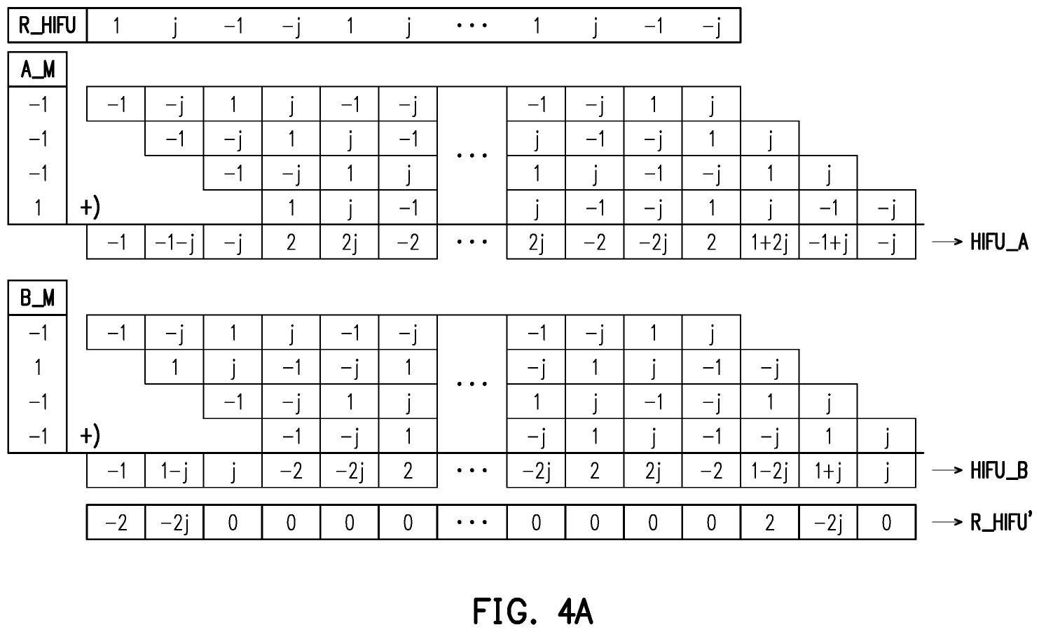

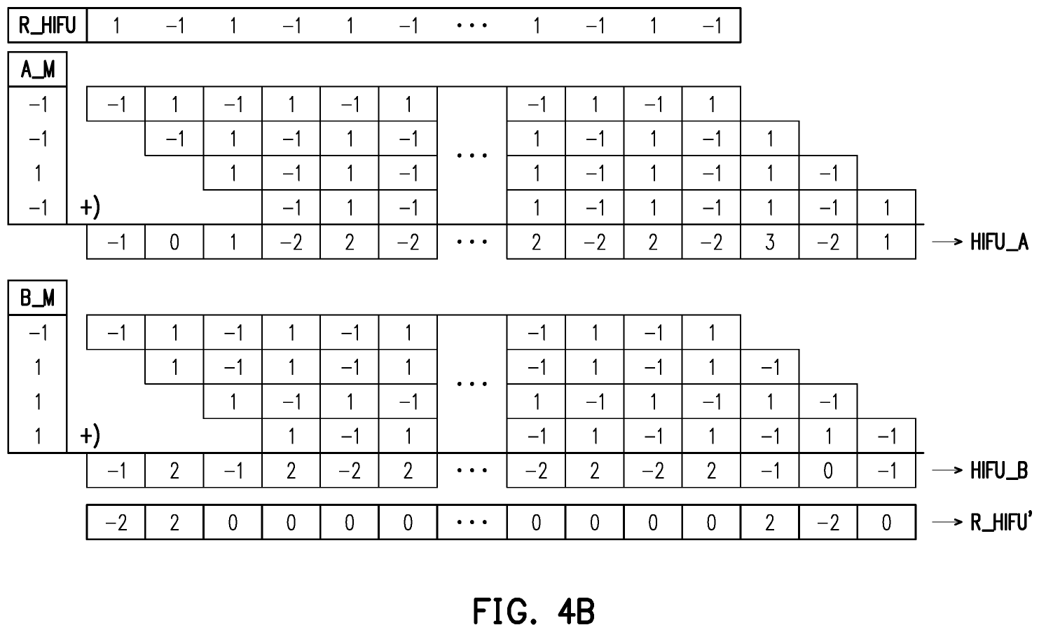

Due to a specific ratio of the Golay code bit period of the imaging signal to the period of the HIFU signal (as shown in formula (2) and formula (3)), a sequence of the HIFU interference signal may be effectively suppressed. FIG. 4A is a schematic diagram illustrating decoding details of the HIFU interference signal in the first embodiment. As shown in FIG. 4A, addition is performed after convolution processing is respectively performed on the HIFU interference signal R_HIFU through the matching filters A_M and B_M. In the present embodiment, the HIFU interference signal R_HIFU is, for example, [1 j −1 −j 1 j . . . 1 j −1 −j], and the matching filters A_M and B_M are, for example, [−1 −1 −1 1] and [−1 1 −1 −1]. The convolution processing may be performed on the HIFU interference signal R_HIFU through the matching filter A_M to obtain an output signal HIFU_A. Similarly, the convolution processing may be performed on the HIFU interference signal R_HIFU through the matching filter B_M to obtain an output signal HIFU_B. Description of the convolution operation is not repeated herein since the convolution operation is well-known to a person of ordinary skill in the art. The output signals HIFU_A and HIFU_B may be added to obtain a suppressed HIFU interference signal R_HIFU′. It may be learned that, compared with the HIFU interference signal R_HIFU, the suppressed HIFU interference signal R_HIFU′ only has a little residual intensity at two ends while the rest is cancelled to zero. With the aforementioned HIFU cancellation, a monitoring ultrasound image can be constructed with a sufficient depth range and a high SIR during the HIFU therapy.

FIG. 4B is a schematic diagram illustrating decoding details of the HIFU interference signal in the second embodiment. As shown in FIG. 4B, similarly, addition is performed after convolution processing is respectively performed on the HIFU interference signal R_HIFU through the matching filters A_M and B_M. In the present embodiment, the HIFU interference signal R_HIFU is, for example, [1 −1 1 −1 −1 . . . 1 −1 1 −1], and the matching filters A_M and B_M are, for example, [−1 −1 1 −1] and [−1 1 1 1]. The convolution processing may be performed on the HIFU interference signal R_HIFU through the matching filter A_M to obtain an output signal HIFU_A. Similarly, the convolution processing may be performed on the HIFU interference signal R_HIFU through the matching filter B_M to obtain an output signal HIFU_B. The output signals HIFU_A and HIFU_B may be added to obtain a suppressed HIFU interference signal R_HIFU′. It may be learned that, compared with the HIFU interference signal R_HIFU, the suppressed HIFU interference signal R_HIFU′ only has a little residual intensity at two ends while the rest is cancelled to zero. With the aforementioned HIFU cancellation, a monitoring ultrasound image can be constructed with a sufficient depth range and a high SIR during the HIFU therapy. In addition, it should be noted that the achievable HIFU interference cancellation of the first embodiment is equivalent to that of the second embodiment.

In the disclosure, the imaging signal P is not limited to only a 4-bit Golay code. In the disclosure, the imaging signal P may be a 4^{N}-bit Golay code, and N may be any non-negative integer. The aforementioned 4^{N}-bit Golay code may be learned by a Golay-paired Hadamard matrix. Taking N=2 as an example, the imaging signal is a 16-bit Golay code, and the matching filters A_M and B_M are also 16 bits. In an extended example of the first embodiment, the matching filter A_M can be [−1 −1 −1 1 −1 −1 1 −1 −1 −1 −1 1 1 1 −1 1], and the matching filter B_M can be [−1 1 −1 −1 −1 1 1 1 −1 1 −1 −1 1 −1 −1 −1]. In an extended example of the second embodiment, the matching filter A_M can be [−1 −1 1 −1 −1 −1 −1 1 −1 −1 1 −1 1 1 1 −1], and the matching filter B_M can be [−1 1 1 1 −1 1 −1 −1 −1 1 1 1 1 −1 1 1]. Similarly, for the 16-bit Golay code, the suppressed HIFU interference signal R_HIFU′ only has a little residual intensity at two ends while the rest is cancelled to zero.

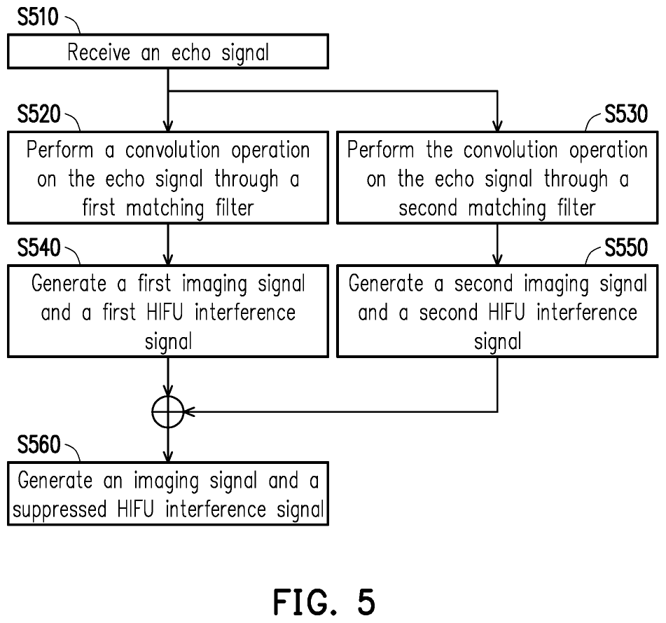

FIG. 5 is a flow chart illustrating steps of a real-time monitoring method of a HIFU therapeutic system. Referring to FIG. 1 and FIG. 5, the real-time monitoring method of the HIFU therapeutic system includes: receiving an echo signal \hat{P} (step S510), where the echo signal \hat{P} includes a HIFU interference signal R_HIFU and an echo signal Â or an echo signal \hat{B}; performing a convolution operation through a matching filter A_M when the echo signal is Â (step S520) and generating a first imaging signal and a first HIFU interference signal after the convolution operation (step S540); performing a convolution operation through a matching filter B_M when the echo signal is \hat{B} (step S530) and generating a second imaging signal and a second

HIFU interference signal after the convolution operation (step S550); and adding above calculation results to obtain an enhanced imaging signal and a suppressed HIFU interference signal (step S560).

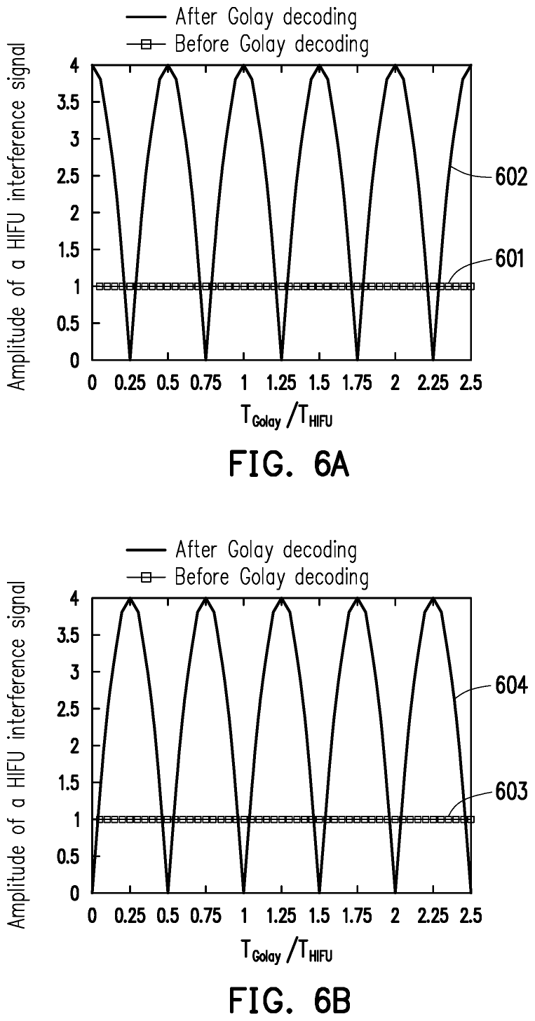

In the following, FIG. 6A and FIG. 6B are used to respectively show suppression effects of the HIFU interference signal in the first embodiment and the second embodiment. FIG. 6A is a schematic diagram illustrating comparison before and after Golay decoding for the first embodiment. Referring to FIG. 6A, the horizontal axis represents the ratio of T_{Golay} to T_{HIFU}, and the vertical axis represents the amplitude of the HIFU interference signal. A line 601 shows an original amplitude of the HIFU interference signal received by the image probe. A curve 602 shows a variation of the amplitude of the suppressed HIFU interference signal after 4-bit Golay decoding at different ratios. It may be learned from FIG. 6A that when the ratio is 0.25, 0.75, or 1.25, that is, when the ratio of T_{Golay} to T_{HIFU} is ¼, ¾, or 5/4, the HIFU interference signal is almost suppressed to zero which corresponds to the optimal effect for HIFU elimination.

FIG. 6B is a schematic diagram illustrating comparison before and after Golay decoding for the second embodiment. Referring to FIG. 6B, similarly, the horizontal axis represents the ratio of T_{Golay} to T_{HIFU}, and the vertical axis represents the amplitude of the HIFU interference signal. A line 603 shows an original amplitude of the HIFU interference signal received by the image probe. A curve 604 shows a variation of the amplitude of the suppressed HIFU interference signal after 4-bit Golay decoding at different ratios. It may be learned from FIG. 6B that when the ratio is 0.5, 1.0, or 1.5, that is, when the ratio of T_{Golay} to T_{HIFU} is 2/4, 4/4, or 6/4, the HIFU interference signal is almost suppressed to zero which corresponds to the optimal effect for HIFU elimination.

In view of the above, according to the disclosure, ultrasonic imaging may be performed by transmitting a Golay code to improve an image signal-to-noise ratio. Further, according to the disclosure, the specific ratio of the Golay code bit period to the period of the HIFU signal (as shown in formula (2) and formula (3)) is further used to eliminate the interference pattern caused by the HIFU signal on an ultrasonic image. Therefore, in the disclosure, real-time ultrasound image guidance with high image quality and full windows is provided, maintain efficiency of a HIFU therapy is maintained as well.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A high-intensity focused ultrasound (HIFU) therapeutic system, comprising:
a first ultrasonic transmitter configured to transmit a high-intensity focused ultrasound therapeutic signal to a target; and
an ultrasonic imaging apparatus comprising:
a second ultrasonic transmitter configured to alternately transmit a first imaging signal and a second imaging signal to the target, wherein the first imaging signal and the second imaging signal form a complementary Golay code pair;
an echo receiver configured to receive a first echo signal corresponding to the first imaging signal, a second echo signal corresponding to the second imaging signal, and an interference signal caused by the high-intensity focused ultrasound therapeutic signal; and
a signal processor configured to perform a decoding operation on the first echo signal, the second echo signal, and the interference signal to generate an imaging signal and a suppressed interference signal.

2. The high-intensity focused ultrasound therapeutic system according to claim 1, wherein both the first imaging signal and the second imaging signal are 4^{N} bits, and N is a positive integer.

3. The high-intensity focused ultrasound therapeutic system according to claim 1, wherein a bit period length of the first imaging signal and a bit period length of the second imaging signal are (2a+1)/4 times a period length of the high-intensity focused ultrasound therapeutic signal, and a is an integer greater than or equal to 0.

4. The high-intensity focused ultrasound therapeutic system according to claim 1, wherein a bit period length of the first imaging signal and a bit period length of the second imaging signal are (2a+2)/4 times a period length of the high-intensity focused ultrasound therapeutic signal, and a is an integer greater than or equal to 0.

5. The high-intensity focused ultrasound therapeutic system according to claim 1, wherein the decoding operation comprises:
performing a convolution operation on the first echo signal through a first matching filter to generate a first operation result;
performing the convolution operation on the second echo signal through a second matching filter to generate a second operation result; and
calculating a sum of the first operation result and the second operation result to generate the imaging signal.

6. The high-intensity focused ultrasound therapeutic system according to claim 5, wherein the decoding operation further comprises:
performing the convolution operation on the interference signal through the first matching filter to generate a third operation result;
performing the convolution operation on the interference signal through the second matching filter to generate a fourth operation result; and
calculating a sum of the third operation result and the fourth operation result to generate the suppressed interference signal.

7. A real-time monitoring method of a high-intensity focused ultrasound (HIFU) therapeutic system, comprising:
transmitting a high-intensity focused ultrasound therapeutic signal to a target;
alternately transmitting a first imaging signal and a second imaging signal to the target, wherein the first imaging signal and the second imaging signal form a complementary Golay code pair;
receiving a first echo signal corresponding to the first imaging signal, a second echo signal corresponding to the second imaging signal, and an interference signal caused by the high-intensity focused ultrasound therapeutic signal; and
performing a decoding operation on the first echo signal, the second echo signal, and the interference signal to generate an imaging signal and a suppressed interference signal.

8. The real-time monitoring method of the high-intensity focused ultrasound therapeutic system according to claim 7, wherein both the first imaging signal and the second imaging signal are 4^{N} bits, and N is a positive integer.

9. The real-time monitoring method of the high-intensity focused ultrasound therapeutic system according to claim 7, wherein a bit period length of the first imaging signal and a bit period length of the second imaging signal are (2a+1)/4 times a period length of the high-intensity focused ultrasound therapeutic signal, and a is an integer greater than or equal to 0.

10. The real-time monitoring method of the high-intensity focused ultrasound therapeutic system according to claim 7, wherein a bit period length of the first imaging signal and a bit period length of the second imaging signal are (2a+2)/4 times a period length of the high-intensity focused ultrasound therapeutic signal, and a is an integer greater than or equal to 0.

11. The real-time monitoring method of the high-intensity focused ultrasound therapeutic system according to claim 7, wherein the decoding operation comprises:
performing a convolution operation on the first echo signal through a first matching filter to generate a first operation result;
performing the convolution operation on the second echo signal through a second matching filter to generate a second operation result; and
calculating a sum of the first operation result and the second operation result to generate the imaging signal.

12. The real-time monitoring method of the high-intensity focused ultrasound therapeutic system according to claim 11, wherein the decoding operation further comprises:
performing the convolution operation on the interference signal through the first matching filter to generate a third operation result;
performing the convolution operation on the interference signal through the second matching filter to generate a fourth operation result; and
calculating a sum of the third operation result and the fourth operation result to generate the suppressed interference signal.
